(19) United States
(12) Patent Application Publication
(10) Pub. No.: US 2006/0004375 A1
(43) Pub. Date: Jan. 5, 2006

(54) Surgical instrument

(76) Inventors: Neil Watkins, Lymm (GB); Magnus Flett, Wakefield (GB); Stefano Alfonsi, Harrogate (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/059,051

(22) Filed: Feb. 16, 2005

(30) Foreign Application Priority Data
Feb. 16, 2004 (GB) 0403363.5

(51) Int. Cl. A61B 17/88; A61F 2/46; A61F 2/36

(52) U.S. Cl. 606/94; 623/23.13

(57) ABSTRACT

An instrument for preparing bone tissue on the head of a bone which provides the ball component of a ball and socket joint, to receive a re-surfacing prosthesis, comprises a hollow shell which can be fitted around the head to define a space around the head, and a flexible seal component which can form a seal between the shell and the neck of the bone below the head. When bone cement within the shell that is subjected to pressure, leakage of the cement from within the shell is minimised by virtue of the seal between the shell and the neck of the bone provided by the flexible seal component. The instrument can be used in a method which involves the application of positive pressure to the cement in the space around the head of the bone, to force it into pores in the bone.

### BACKGROUND OF THE INVENTION

This invention relates to an instrument for preparing bone tissue on the head of a bone which provides the ball component of a ball and socket joint to receive a re-surfacing prosthesis.

When ball and socket joints such as hip joints and shoulder joints are damaged, it is common to replace the entire joint with a joint prosthesis. In the case of a damaged hip joint, replacement involves resection of the proximal femur and implantation of the femoral component of an orthopaedic joint, which includes a stem part which can be received in the intramedullary canal, and a head part with a convex bearing surface. The patient's acetabulum is prepared to receive the acetabular component of the joint prosthesis, which provides a concave bearing surface to articulate with the bearing surface on the femoral component. Frequently, bone cement is used to affix the components of the prosthesis within their respective prepared bone cavities.

When the condition of the femoral bone tissue is generally good, it can be desirable to retain much of the proximal femur. Techniques have been developed in which the femoral head is fitted within a hollow resurfacing shell. The resurfacing shell has a convex outer surface which is highly polished which enables it to act against the hollow bearing surface of an acetabular component. Such techniques are referred to as Articular Surface Replacement techniques. They have the advantage that the quantity of bone which has to be removed from the head of the bone is only small. A tool which can be used to prepare the head in this way is disclosed in International patent application no. GB03/04303.

A bone cement can be used to ensure that an appropriate bond is formed between a resurfacing shell and the prepared head of a femur or other bone. Bone cements which are used in such techniques tend to have a relatively low viscosity. They are used in a technique in which a quantity of the cement is provided within the resurfacing shell which is then fitted on to the prepared head. Reception of the head within the shell causes the cement to be displaced. Application of pressure on to the shell causes the cement to penetrate the porous structure of the head. Excess cement is made to exude from within the shell. Low viscosity cement is used in this way to facilitate penetration of the cement into the porous bone structure. Furthermore, use of a low viscosity cement ensures that the cement can be displaced when the shell is fitted on to the head of the bone, so that the shell can be properly seated on the head.

When pressure applied to a shell is relied on to cause low viscosity cement to penetrate the porous surface of a bone, it has been found that penetration can be achieved satisfactorily in the region around the pole of the head, but that penetration in the surfaces which face generally outwardly (radially relative to the polar axis) is not always satisfactory, and penetration at the interface between the head of the bone and the implant at its periphery can be negligible. This can lead to undesirable side effects such as loosening of the prosthesis.

### SUMMARY OF THE INVENTION

The present invention provides an instrument for preparing bone tissue on the head of a bone which provides the ball component of a ball and socket joint, to receive a re-surfacing prosthesis, which comprises a hollow shell which can be fitted around the head to define a space around the head, and a flexible seal component which can form a seal between the shell and the neck of the bone below the head, so that when bone cement within the shell is subjected to pressure, leakage of bone cement from within the shell is minimised by virtue of the seal between the shell and the neck of the bone provided by the flexible seal component.

The instrument of the invention can be used to prepare a cement mantle on a prepared head of a bone in such a way that the cement will penetrate the surface of the bone, prior to fitting a resurfacing shell on to the head, over the cement mantle. The use of a shell to apply pressure to the cement mantle can enable pressure to be applied to the mantle more uniformly than if the resurfacing head itself is relied on to apply the pressure, while being positioned on the head of the bone. This can enable penetration of the cement into the porous structure of the bone to be more uniform, including into surfaces of the bone which face generally radially outwardly relative to the polar axis of the head of the bone as well as into the surfaces around the pole. This can enable the interface between the bone and the resurfacing shell to withstand torsional forces more effectively. It can provide greater resistance to ingress of foreign particles at the interface between the head of the bone and the implant at its periphery can be negligible. It can also provide a more uniform distribution of bone cement around the head of the bone; in particular, the tendency for cement to collect at the pole can be reduced. This is beneficial because localised accumulation of bone cement can give rise to a higher than normal temperature as the cement cures, possibly lead to damage of the bone tissue in some circumstances.

Preferably, the shell comprises at least two part shells which can be assembled together to define the space around the head. Part shells can be connected to one another by means of a hinge, allowing one part to be pivoted relative to the other part. Part shells can be connected to one another by means of a channel member, which can be fitted over rails at the aligned edges of the part shells by sliding. Part shells can be connected to one another by means of one or more clamps at adjacent aligned edges.

Part shells can be held in a closed position, enclosing the head of the bone, by means of handles. This is particularly appropriate when the bone cement will harden sufficiently over a relatively short period of time, for example up to 10 minutes or so, to allow the shell to be removed from around the cement mantle.

The shell can be provided by a rigid material so that the shell is not deformed significantly when subjected to pressures normally encountered in the pressurisation of bone cement. For example, the shell can comprise a sheet-like wall which is formed from a material which will not deform significantly. The wall can be reinforced by one or more reinforcing webs, generally on the external surface of the wall. When the wall of the shell is sheet-like, the shell can be fabricated from sheet, for example by welding or other bonding techniques. A shell with a shell-like wall could also be fabricated by other techniques such as casting or moulding.

Suitable materials can include polymers and metals such as those which are commonly used in surgical instruments. Suitable polymers might include certain polycarbonates, polyesters and polyamides. Suitable metals might include certain stainless steels.

The thickness of the wall of the shell will be selected to ensure that the shell has the structural properties which are required for it to perform satisfactorily. The thickness that is required will also depend on the material which is used, consistent with it having the required structural properties.

The shell can comprise a flexible wall member, and a supporting frame positioned outside the wall member. For example, the shell can comprise a resiliently deformable membrane which can be located around the prepared head. The deformability of the membrane can enable it to conform to the shape and size of the head, to provide an appropriately shaped cement mantle around the head.

Preferably, the surface of the inner wall of the shell is provided by a release material so that adhesive forces between bone cement materials and the inner wall of the shell are minimal. This allows the shell to be removed cleanly from the cement mantle on the head of the bone. Examples of suitable release materials for use with available bone cement materials will be known to the skilled reader. Silicone rubber based materials are suitable for many applications. The release material can be provided as a surface layer of coating on the inner wall of the shell. The wall of the shell can consist essentially of the release material.

Commonly, a resurfacing shell has a stem extending along the polar axis, which is received in a bore in the head of the bone along the polar axis of the head. The stem helps to locate the shell on the head. Such a stem is frequently tapered inwardly along its length from the root where it is connected to the shell to its distal tip. Preferably, the instrument includes a stem hole occluder. A stem hole occluder can be positioned in a bore in the bone, to prevent passage of bone cement into the bore.

A stem hole occluder can be provided as a component which is separate from the shell and which is fitting into a bore in the head of the bone before the head of the bone is located within the shell.

A stem hole occluder can be connected to the shell, at least during the period in which the shell is closed around the head of the bone, so that the occluder can be removed from within the bore in the bone at the same time as the shell is removed from around the bone.

In another aspect, the invention provides apparatus for use in resurfacing of the head of a bone which provides the ball component of a ball and socket joint, which comprises an instrument as discussed above and a quantity of a bone cement.

In a further aspect, the invention provides a method of preparing bone tissue on the head of a bone which provides the ball component of a ball and socket joint, to receive a re-surfacing prosthesis, which comprises:
a. shaping the head of the bone,
b. fitting a mould around the head of the bone to define a space within the mould around the head of the bone,
c. applying bone cement material to the head of the bone,
d. applying positive pressure to the cement material within the said space to force the cement material into pores within the structure of the bone, and
e. removing the mould from around the head of the bone.

The instrument of the invention can be used with high viscosity bone cements which do not flow readily. Such bone cements have a dough like consistency and are substantially self-supporting so that a quantity of the cement does not flow significantly when left to stand for a period of about 10 minutes. This allows a cement to be manipulated when used in a surgical procedure, after it has been mixed. Such a cement can be applied to the head of bone manually: use of a syringe to apply the cement is generally not possible because of its viscosity. Generally, the viscosity of a high viscosity cement, before application to the surface of the bone, will be at least about 10⁶ cp, preferably at least about 1.5×10⁶ cp.

The instrument of the invention can be used with low viscosity bone cements which flow readily. Such bone cements can be applied to the head of a bone using a syringe. Preferably, the shell has a port in it, through which bone cement can be injected into the space between the shell and the surface of the bone, especially using a syringe. The apparatus of the invention can include a tool which can fit into the port for delivery of bone cement into the said space, for example a syringe.

Generally, the viscosity of a low viscosity cement, before application to the surface of the bone, will be not more than about 10⁶ cp, preferably not more than about 0.75×10⁶ cp.

The viscosity of a bone cement can be measured using a viscometer. An example of a suitable instrument is that available under the trade name Brookfield RVDV-III. The reactive bone cement components (generally a powder and a liquid) should be at 23° C. before mixing, and should be thoroughly mixed (in line with manufacturers' instructions) before the viscosity measurements are taken.

When the instrument has a port in it for injection of bone cement, the port can be provided at about the polar axis. When the instrument includes a stem hole occluder, it can be associated with the port in the shell. For example, the stem hole occluder can be connected to the shell around the port. The stem hole occluder can include an upstanding flange which can protrude through the port, which can be gripped after the shell has been removed from around the bone to withdraw the stem hole occluder from within the bore in the bone. The flange can have laterally directed openings to allow bone cement that is injected through the port to flow throughout the space between the shell and the head of the bone.

The flexible seal component can comprise a dam formed from a resiliently deformable material. The seal component should by sufficiently long to allow it to extend completely around the neck of the bone, below the head. The seal should be capable of engaging the bone tightly around the neck. The seal component can comprise an elastomeric material, for example a silicone rubber. The seal component can be formed from a single piece of silicone rubber with a liner for a shell, or a shell which comprises a flexible wall member and a supporting frame positioned outside the wall member. The seal component might have a thicker wall compared with a liner for a shell or the flexible wall member within a supporting frame. The material and dimensions of the seal component will be selected according to the bone cement material that is used, the pressures that are expected to be generated within the shell, the size of the head and neck of the bone and similar factors.

Preferably, the instrument includes a clamp by which the seal component can be clamped tightly on to the bone around the neck so that elevated pressure can be generated within the space between the shell and the head of the bone. The clamp can be closed by means of a screw threaded fastener, for example a combination of threaded bolt and a nut. The clamp might be closed by means of a spring loaded clamp.

Generally, the resurfacing shell which is fitted on to the head of the bone will define an internal cavity which is rotationally symmetrical so that the cross-sectional shape of the cavity on the plane containing the polar axis remains substantially unchanged around the shell. Generally the cavity will comprise a polar surface which is approximately planar, perpendicular to the polar axis, and a side wall which is inclined to the polar axis. Preferably, the included angle between the side wall and the polar axis at the edge of the side wall where it intersects the polar surface is at least about 5°, for example about 10°. The included angle will generally be less than about 20°.

The head of the bone can be prepared using a reamer. A tool which can be used to prepare the head in this way is disclosed in International patent application no. GB03/04303.

The shell in the instrument of the invention will define a cavity which has a shape similar to the shape of the cavity in the resurfacing shell which is to be used, so that the shape of the cement mantle which is created on the head of the bone is similar to the cavity within the resurfacing shell. Generally, the cavity in-the shell of the instrument will be slightly bigger than the cavity in the resurfacing shell. For example, the radius or depth of the cavity in the shell of the instrument might be bigger than the radius or depth of the cavity in the resurfacing shell by at least about 0.7 mm, preferably at least about 1.0 mm, more preferably at least about 1.5 mm. This can provide for a mantle around the head of the bone of a predetermined thickness.

Preferably, the shell of the invention includes at least one locator within it, which extends inwardly from the internal surface of the shell, to contact the bone and to locate the shell relative to the bone. The locator is intended to contact the surface of the resected bone so that the shell is located relative to the surface, along the axis of the bone or transversely relative to it, or preferably both. Preferably, the shell includes a plurality of locators. For example a plurality of locators can extend inwardly from the side wall of the shell to locate the shell transversely relative to the bone. Preferably, there are at least three such locators. They might be provided, for example, at or close to the upper edge or the lower edge or both of the side wall of the shell. One or more locators can also be provided to extend towards the upper surface of the bone, to locate the shell along the axis of the bone.

Preferably the or each locator has a small cross-section so as to minimise its interference with the flow of cement within the shell. For example, locators can have the configuration of small pins. The cross-sectional shape and size of the pins will depend on factors such as the size of the shell, the intended thickness of the cement mantle (and therefore the length of the pins), the material from which the pins are formed and so on. When the shell is provided by a rigid material which is not deformed significantly under pressure, such as a metal or a polymer, the pins can be formed with the shell, for example by a moulding or casting process. When the shell comprises a flexible wall member, and a supporting frame positioned outside the wall member, the pins can be formed with the supporting frame, so that they extend through holes formed in the wall member.

Preferably, the length of the or each locator is at least about 2 mm, more preferably at least about 3 mm, for example at least about 4 mm. Preferably, the length of the or each locator is not more than about 8 mm, more preferably not more than about 6 mm.

Preferably, the shell has openings in its wall for the relief of pressure within it. The openings should only be small so that they do not limit the pressure which can be generated within the shell. For example, the shell might have two openings in its wall, each with an area of not more than about 3 mm².

Preferably, the bone cement material is applied to the head of the bone before the mould is fitted to the head of the bone. Pressure can then be applied to the cement material by closing the mould around the head of the bone.

Preferably, the mould is fitted to the head of the bone before the bone cement material is applied, so that the bone cement material is supplied to the said space within the mould around the head of the bone. It can be particularly preferred that pressure is applied to the cement material by injecting the material into the space within the mould. The method can include the step of applying a suction force to the space within the mould around the head of the bone to encourage the flow of bone cement into the said space.

When the bone has a bore formed in it aligned approximately with the axis of the head of the bone, it can be preferred for the method to include the step of positioning an occluder device in the bore to prevent passage of the bone cement into the bore.

Preferably, the method includes the step of forming a seal between the mould and the bone, in the neck region of the bone.

### INTRODUCTION TO THE DRAWINGS

FIG. 1 is a cross-section through a femoral head which has a resurfacing shell fitted to it.

FIG. 2 shows steps in a method of fitting a resurfacing shell to a femoral head.

FIG. 3 is a side view of a first embodiment of the instrument of the invention.

FIG. 4 is a sectional elevation through a second embodiment of the instrument of the invention, in use.

FIG. 5 is a sectional elevation through a third embodiment of the instrument of the invention, in use.

### DETAILED DESCRIPTION OF THE INVENTION

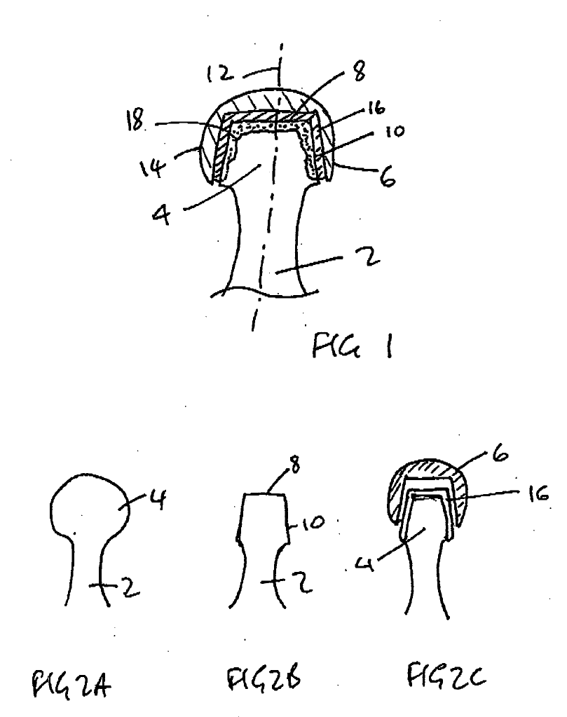

Referring to the drawings, FIG. 1 shows the neck 2 and head 4 of a femur to which a resurfacing shell 6 has been affixed using a bone cement. It should be noted that the present invention can be used in relation to bones other than the femur, including for example the humerus.

The head 4 of the femur has been prepared to receive the resurfacing shell 6 by reaming it to form a planar top surface 8 and a side surface 10 which is inclined to the axis 12 of the head and neck so that the angle between the side surface and the axis is about 7°.

The resurfacing shell 6 defines an internal cavity in which the head of the femur is fitted. The shape of the cavity corresponds to the shape of the reamed head, except that it is slightly larger in each dimension (radius, depth) by about 2 mm. The resurfacing shell 6 has a spherical external surface 14 which is polished.

A mantle of a bone cement 16 is provided between the surface of the bone and the internal surface of the cavity in the resurfacing shell.

For secure fixation of the resurfacing shell on to the prepared bone, it is preferred that the bone cement should penetrate the porous bone tissue of the head as shown at 18 in FIG. 1. This can be achieved be applying pressure to the bone cement while it is fluid. It is preferred that the bone cement should penetrate the surface of the head in the side surface regions as well as the top surface region.

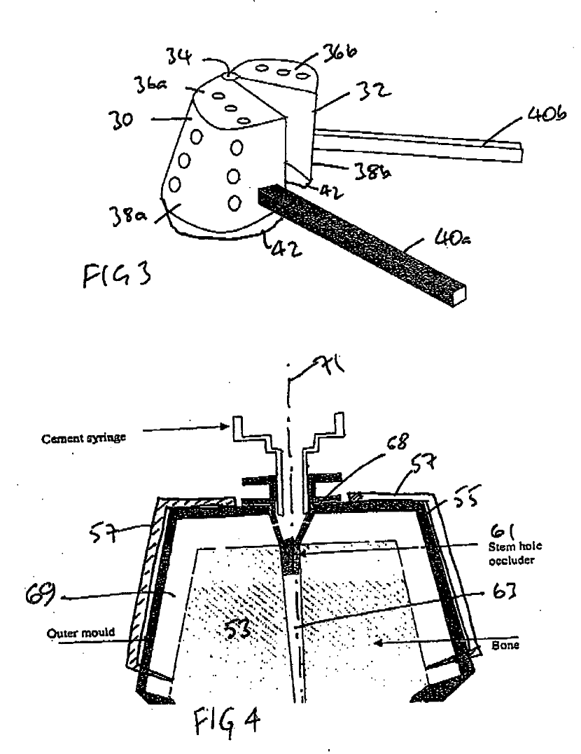

FIG. 2a shows the head 4 and neck 2 region of a femur before bone tissue is resected.

FIG. 2b shows the head after it has been resected, for example using a reamer of the type disclosed in International patent application no. GB03/04303, so that it has a planar top surface 8 and a side surface 10.

FIG. 2c shows the head with a mantle 18 of a bone cement formed around it, prior to a resurfacing shell 6 being fitted over the head.

FIG. 3 shows an instrument which can be used to apply pressure to a mantle of bone cement which is provided around a prepared head. The instrument comprises two shell parts 30, 32. The shell parts are connected to one another along aligned edges by means of a hinge 34 so that they can be moved relative to one another between an open configuration as shown in FIG. 3 and closed configuration. In the closed configuration, the shell parts between them define a cavity which has the shape of a truncated cone which has a top wall 36a, 36b and a side wall 38a, 38b, and a circular cross-section. The shell parts each define a part of that cavity with one of the cavity parts being a mirror image of the other cavity part.

Each of the cavity parts has a handle 40a, 40b by which the shell parts can be moved relative to one another, and held in the closed configuration.

The instrument is open at its base. A flexible seal component 42 depends from the base, which can be wrapped around the neck of the femur and clamped there by means of a clamp.

The instrument is formed from stainless steel. The cavity is lined with a silicone rubber. The wall of the instrument has holes 44 extending through it to allow air within the cavity to escape.

The instrument shown in FIG. 3 finds particular application with bone cements which are relatively viscous, for example with a viscosity of at least about 10⁶ cp, and which are applied to a bone with a dough-like consistency.

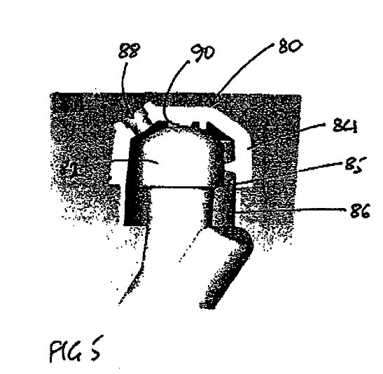

FIG. 4 shows an instrument which can be used to form a mantle of a bone cement around the head 53 of a femur or other bone, using a bone cement which has a low viscosity and can flow, in particular so that it can be dispensed from a syringe. The instrument includes a shell which comprises a silicone rubber wall member 55 and a supporting frame 57 which can be fitted around the wall member to support it against internal forces. The wall member is formed as a single piece with a seal member 59.

The supporting frame provides a plurality of locator pins 60 which extend through the wall member, in use to contact the surface of the femoral head 53 so as to locate the shell laterally relative to the head.

The instrument includes a stem hole occluder 61 which can be fitted into an axial bore 63 in the head 53 of the femur.

The wall member has a port 65 formed in it which has a socket 67 to receive the nozzle of a syringe from which low viscosity bone cement can be dispensed. The socket is formed integrally with the stem hole occluder. There are side openings 68 in the socket which allow bone cement to flow into the space 69 between the wall member 55 and the surface of the head 53. The socket and the stem hole occluder between them provide control over the position of the shell relative to the head of the femur along the axis 71 of the head.

In use, the femoral head is prepared by forming the top and side wall surfaces, as discussed above in relation to FIG. 2b. The wall member 55 is positioned around the head so that the seal member 59 surrounds the neck 73 and so that the stem hole occluder 61 is received in the bore 63 in the femur. The supporting frame 57 is positioned around the wall member. A clamp 75 is positioned around the seal member.

The nozzle 77 of a cement mixing and delivery syringe is received in the socket 67. Pressure is applied to the syringe to displace bone cement from within it, into the space 69 between the wall member 55 and the surface of the head 53. continued application of force to the bone cement using the syringe causes the pressure within the wall member to increase. This can force bone cement into pores in the surface of the bone.

The clamp, supporting frame, and wall member are removed from the bone cement after it has hardened partially, to the extent that it is sufficiently self supporting for it not to drop from around the head to an unacceptable degree. A resurfacing shell can then be fitted to the head, over the cement mantle.

FIG. 5 shows an instrument 80 which can be used to form a mantle of a bone cement around the head 82 of a femur or other bone, using a bone cement which has a low viscosity and can flow, in particular so that it can be dispensed from a syringe.

The instrument comprises a shell 84 which is formed from a rigid polymer, having a lining 85 of a release material such as a silicone polymer. The shell 84 has a skirt 86 depending from it which is formed from the silicone polymer, which is flexible and can be sealed against the neck of the femur (or other bone), for example, by means of a strap which can be tightened around the neck. The skirt can be formed as a single component with the shell lining 85.

The shell has an inlet port 88 which is located away from the axis of the shell so that the angle between the axis and a line extending from the port to the centre of the head of the femur is at least about 25°, preferably at least about 30°. The said angle is preferably not more than about 65°, more preferably not more than about 55°.

The shell can have a stem hole occluder 90 formed integrally within it (shown in dotted outline in FIG. 5), so that cement injected into the space within the shell, between the shell and the surface of the bone, does not flow into the stem hole. The stem hole occluder can also help to locate the shell centrally on the head of the bone.

Optionally, the shell can have a suction port through which a suction pump can be connected to remove air and blood from the space within the shell. The suction port can communicate with the stem hole to remove fluids from that region.

What is claimed is:

1. A method of preparing bone tissue on the head of a bone which provides the ball component of a ball and socket joint, to receive a re-surfacing prosthesis, which comprises:
a. shaping the head of the bone,
b. fitting a mould around the head of the bone to define a space within the mould around the head of the bone,
c. applying bone cement material to the head of the bone,
d. applying positive pressure to the cement material within the said space to force the cement material into pores within the structure of the bone, and
e. removing the mould from around the head of the bone.

2. A method as claimed in claim 1, in which the bone cement material is applied to the head of the bone before the mould is fitted to the head of the bone.

3. A method as claimed in claim 2, in which pressure is applied to the cement material by closing the mould around the head of the bone.

4. A method as claimed in claim 1, in which the mould is fitted to the head of the bone before the bone cement material is applied, so that the bone cement material is supplied to the said space within the mould around the head of the bone.

5. A method as claimed in claim 4, in which pressure is applied to the cement material by injecting the material into the space within the mould.

6. A method as claimed in claim 4, which includes the step of applying a suction force to the space within the mould around the head of the bone to encourage the flow of bone cement into the said space.

7. A method as claimed in claim 1, in which the bone has a bore formed in it aligned approximately with the axis of the head of the bone, and in which the method includes the step of positioning an occluder device in the bore to prevent passage of the bone cement into the bore.

8. A method as claimed in claim 1, which includes the step of forming a seal between the mould and the bone, in the neck region of the bone.

9. An instrument for preparing bone tissue on the head of a bone which provides the ball component of a ball and socket joint, to receive a re-surfacing prosthesis, which comprises a hollow shell which can be fitted around the head to define a space around the head, and a flexible seal component which can form a seal between the shell and the neck of the bone below the head, so that when bone cement within the shell is subjected to pressure, leakage of bone cement from within the shell is minimised by virtue of the seal between the shell and the neck of the bone provided by the flexible seal component.

10. An instrument as claimed in claim 9, in which the shell comprises at least two part shells which can be assembled together to define the space around the head.

11. An instrument as claimed in claim 9, in which the shell is provided by a rigid material so that the shell is not deformed significantly when subjected to pressures normally encountered in the pressurisation of bone cement.

12. An instrument as claimed in claim 9, in which the shell comprises a flexible wall member, and a supporting frame positioned outside the wall member.

13. An instrument as claimed in claim 9, in which the surface of the inner wall of the shell is provided by a release material, preferably a silicone rubber.

14. An instrument as claimed in claim 9, which includes a stem hole occluder which can be positioned in a bore in the bone, to prevent passage of bone cement into the bore.

15. An instrument as claimed in claim 9, which includes at least one locator within it, which extends inwardly from the internal surface of the shell, to contact the bone and to locate the shell relative to the bone.

16. An instrument as claimed in claim 15, which includes at least three locators which extend inwardly from the side wall of the shell, to locate the shell relative to the bone transversely relative to the axis of the head.

17. An instrument as claimed in claim 9, in which the shell has openings in its wall for the relief of pressure within it.

18. An instrument as claimed in claim 9, in which the shell has an inlet port in it, through which bone cement can be injected into the space between the shell and the surface of the bone.

19. An instrument as claimed in claim 18, in which the inlet port is located away from the axis.

20. An instrument as claimed in claim 18, which includes a tool which can fit into the port for delivery of bone cement into the said space.

21. An instrument as claimed in claim 9, in which the shell has a suction port in it through which a negative pressure can be applied to the space between the shell and the surface of the bone.

22. An instrument as claimed in claim 21, which has a pump connected to it for applying a suction force to the space between the shell and the surface of the bone.

23. Apparatus for use in resurfacing of the head of a bone which provides the ball component of a ball and socket joint, which comprises an instrument as claimed in claim 1 and a quantity of a bone cement.

24. Apparatus as claimed in claim 23, in which the viscosity of the cement, before application to the surface of the bone, is at least about 10⁶ cp.

25. Apparatus as claimed in claim 23, in which the viscosity of the cement, before application to the surface of the bone, is not more than about 10⁶ cp.

26. Apparatus as claimed in claim 23, which includes a resurfacing shell which has a polished external surface which can function as a bearing surface, and which defines an internal cavity in which the head of the bone can be received.
